(19) United States
(12) Patent Application Publication
(10) Pub. No.: US 2018/0049879 A1
(43) Pub. Date: Feb. 22, 2018

(54) BONE CAPTURING KEEL DESIGN FOR ORTHOPEDIC IMPLANTS

(71) Applicant: Arthrex, Inc., Naples, FL (US)

(72) Inventors: Zachary Ingwer, Boca Raton, FL (US); Sergio DaSilva, Naples, FL (US); Alex Bautsch, Naples, FL (US)

(21) Appl. No.: 15/243,855

(22) Filed: Aug. 22, 2016

(51) Int. Cl. A61F 2/30

(52) U.S. Cl. CPC A61F 2/30749; A61F 2310/00023; A61F 2002/30576

(57) ABSTRACT

A keel for an orthopedic implant with a tray portion. The keel has a wall having a posterior end, an anterior end, and sides that extend from a surface of the tray portion. The wall is at least partially upraised away from the surface of the tray portion at its posterior end to define a posterior keel opening. The anterior end may either merge with the surface of the tray portion or can be spaced away to form an anterior keel opening. The keel can slant downwardly from the posterior end to the anterior end by about 10 to 20 degrees. The wall can have two spaced apart flat side walls and a flat top wall, or can be a continuous curved wall. A bone ingrowth space is defined between the wall and the tray portion. Openings can optionally be formed in the wall for additional bone ingrowth.

### BACKGROUND

The disclosure relates to orthopedic implants, and more particularly, to orthopedic implants with keels.

For the most optimal surgical outcome, orthopedic implants must be accurately and reliably secured to the implantation site. The orthopedic implant will ideally include features to provide both immediate and long term securement and stabilization of the implant at the implant site, so that the patient can begin rehabilitation and return to semi-normal activities quickly and without delay, and so that the orthopedic implant will remain in place over the life of the implant.

### SUMMARY

The present disclosure relates to a keel for an orthopedic implant, which keel extends outwardly from a bone contacting platform of the orthopedic implant, with the keel designed to be implanted into a patient's bone in order to help secure the implant to the bone and prevent movement of the implant relative to the bone.

In an exemplary embodiment, the keel has an outer wall that extends from the bone contacting platform and defines a cavity with a volume between the outer wall of the keel and the bone contacting platform wherein the cavity allows for bone ingrowth into the cavity of the keel.

In another exemplary embodiment, the keel has an outer wall that extends from the bone contacting platform, which keel has an open leading mouth and wherein a trailing end of the outer wall merges into the bone contacting platform, the outer wall thus defining a sloped, open mouthed cavity between the outer wall of the keel and the bone contacting platform, wherein the open mouth cavity allows for bone ingrowth into the tunnel of the keel.

In yet another exemplary embodiment, the keel has an outer wall that extends away from the bone contacting platform, which keel has an open leading mouth and an open trailing mouth which define a tunnel between the outer wall of the keel and the bone contacting platform wherein the tunnel allows for bone ingrowth into the tunnel of the keel.

In a further exemplary embodiment, the keel has a curved outer wall that extends away from the bone contacting platform, which keel has an open leading mouth and an open trailing mouth which define a tunnel between the outer wall of the keel and the bone contacting platform wherein the tunnel allows for bone ingrowth into the tunnel of the keel, wherein the open leading mouth may be the same size as the open trailing mouth, or the open leading mouth may be larger than the open trailing mouth.

In still yet a further exemplary embodiment, the keel has an outer wall with two side walls and a top wall, which outer wall extend from the bone contacting platform, which keel has an open leading mouth and an open trailing mouth, which open leading mouth and open training mouth define a tunnel between the outer wall of the keel and the bone contacting platform, wherein the tunnel allows for bone ingrowth into the tunnel of the keel, wherein the open leading mouth may be the same size as the open trailing mouth, or the open leading mouth may be larger than the open trailing mouth. Alternately, instead of having an open trailing mouth, a trailing end of the keel can merge into the bone contacting platform, in which case an open mouth cavity will be formed in the keel. As a further alternative, side windows can be formed on the two side walls to allow for bone growth ingress through the side windows. In still another alternative, protruding barbs can be formed to extend outwardly from the two side walls to provide for further fixation of the keel to the implantation site.

These and other features are described below.

### BRIEF DESCRIPTION OF THE DRAWINGS

FIG. 1A is a perspective view of a prior art implant tray platform and keel, the keel having an arrow-shaped head on a vertical support rib spaced away from the implant tray platform.

FIG. 1B is a detail perspective view showing of the keel of FIG. 1A.

FIG. 1C is a cross-sectional view through view lines 1C-1C of FIG. 1A.

FIG. 2 is perspective view of an exemplary keel shown on an implant tray platform.

FIG. 3A is perspective view of another exemplary keel shown on an implant tray platform.

FIG. 3B is a detail perspective view showing of the keel of FIG. 3A.

FIG. 3C is a cross-sectional view through view lines 3C-3C of FIG. 3A.

FIG. 4 is a diagrammatic cutaway side view showing the prior art implant tray platform and keel with a bearing portion attached, with the bearing portion being subjected to an anterior force and with the keel resisting rocking of the art implant tray platform and keel.

FIG. 5 is a diagrammatic cutaway side view showing the implant tray platform and keel of FIG. 3A with a bearing portion attached, with the bearing portion being subjected to an anterior force and with the keel resisting rocking of the art implant tray platform and keel.

FIG. 6 is a detail perspective view showing another embodiment of a keel with multiple side windows in its side walls.

FIG. 7 is a detail perspective view showing yet another embodiment of a keel with barbs formed on its side walls.

FIGS. 8A-C are a posterior perspective view, top view, and posterior end view, respectively, of another exemplary embodiment of a keel.

FIGS. 9A-C are a posterior perspective view, top view, and posterior end view, respectively, of another exemplary embodiment of a keel.

FIGS. 10A-C are a posterior perspective view, top view, and posterior end view, respectively, of another exemplary embodiment of a keel.

FIGS. 11A-C are a posterior perspective view, top view, and posterior end view, respectively, of another exemplary embodiment of a keel.

FIGS. 12A-C are a posterior perspective view, top view, and posterior end view, respectively, of another exemplary embodiment of a keel.

### DETAILED DESCRIPTION

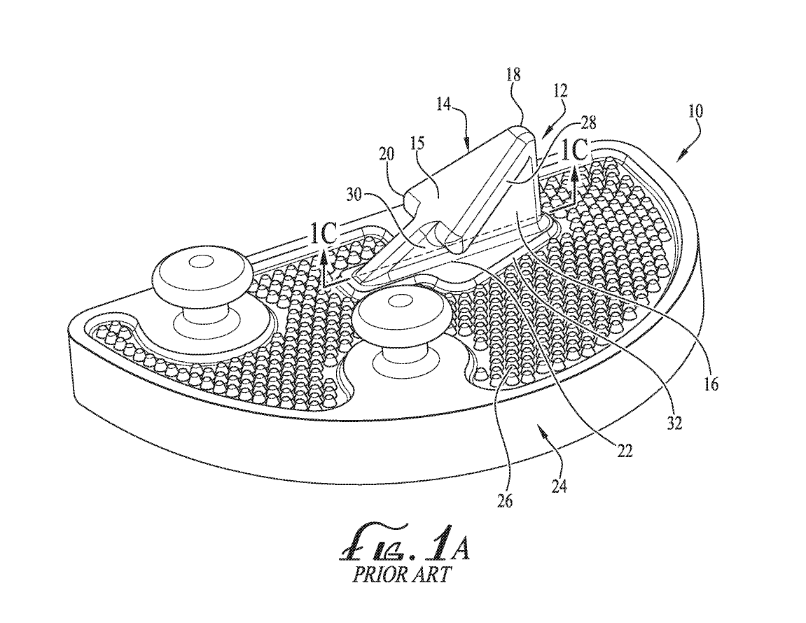

Turning first to FIGS. 1A, 1B, and 1C there are shown, respectively, a perspective view of a prior art implant tray platform with keel 10, a detail view of the keel 12 portion, and a cross-sectional view of FIG. 1B through view lines 1C-1C. The keel 12 has an arrow-shaped head 14 on a vertical support rib 16. The arrow-shaped head 14 has a narrowed posterior end 18 and a wider anterior end 20 with a width W, and has a length of L. The vertical support rib 16 is extends away from a bone facing surface 26 of an implant tray platform 24. The vertical support rib 16 has a lower edge 22 that can extend from a base portion 32, or can directly extend from the bone facing surface 26 of the implant tray platform 24. The arrow-shaped head 14 of the keel 12 has a thickness Hₜ and a perimeter edge 28. The arrow-shaped head 14 has an outwardly facing surface 15 and an inwardly facing surface 17. A portion of the vertical support rib 30 beyond the anterior end 20 slants down and merges into the base portion 32, or can directly extend from the bone facing surface 26 of the implant tray platform 24. Referring to FIG. 1B, at its wider anterior end 20, the outwardly facing surface 15 and inwardly facing surface 17 of arrow-shaped head 14 are spaced distances Hₑₐ and Hᵢₐ, respectively, from the base portion 32, and at its narrower posterior end 18, the outwardly facing surface 15 and inwardly facing surface 17 of arrow-shaped head 14 are spaced distances Hₑₚ and Hᵢₚ, respectively, from the base portion 32. This prior art keel has its arrow-shaped head 14 angled up by angle a about 30 degrees from the anterior to the posterior relative to the base portion 32. Referring to FIG. 1C, there is shown an underside view of the keel 12. The arrow-shaped head 14 is divided by the vertical support rib 16 into two head halves 36A and 36B, which because of the space taken up by the vertical support rib 16, have a smaller surface area than a surface area of the outwardly facing surface 15 of the arrow-shaped head 14. The portion of the vertical support rib 30 forward the anterior end 20 of the arrow-shaped head 14 is not surrounded by any portion of the head. If an imaginary plane is projected straight down from around the perimeter edge 28 of the arrow-shaped head 14 to the base portion 32, the volume between that imaginary line, taking out the volume of the vertical support rib 16, defines a potential bone volume BV that can fill the space under the arrow-shaped head 14. By way of example, with a prior art keel 12 having a keel head thickness Hₜ of 0.0787″ (2.0 mm), a length L of 0.394″ (10.0 mm), an anterior width W of 0.300″ (7.6 mm), a vertical support rib 16 thickness of 0.0787″ (2.0 mm), a distance Hᵢₐ of 0.63 (1.6 mm) from the inwardly facing surface of wider anterior end 20 of the arrow-shaped head 14 to the base portion 32, with the distance Hᵢₚ of 0.300″ (7.6 mm) from the inwardly facing surface of narrow posterior end 20 of the arrow-shaped head 14 to the base portion 32, and with the arrow-shaped head 14 angled about 30 degrees up from anterior to posterior, the cross-sectional surface area of the bottom of the arrow-shaped head 14 would be about 0.0386 square inches (24.9 mm²), and the potential bone volume BV would be about 0.00372 cubic inches (60.9 mm³). The relevance of the surface area of the bottom surface 17 of the arrow-shaped head 14 and of the potential bone volume BV will be discussed further below.

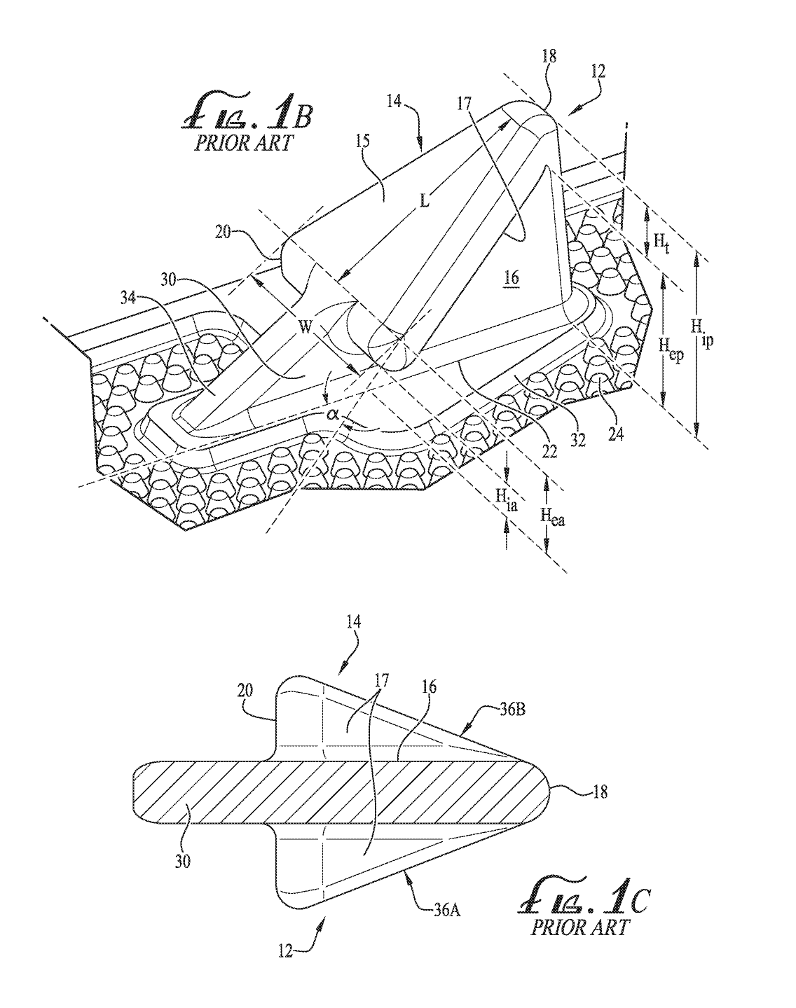

FIG. 2 is a perspective view of an exemplary implant tray platform with keel 40. It includes an implant tray platform portion 41 and a keel 42 extending from a surface 58 of the implant tray platform portion 41. The keel 42 has a top wall 44 and two opposite side walls 46 and 48 that extend from their bottom edges from the tray platform portion 41, a posterior end 50 and an anterior end 52, wherein the posterior end 50 is raised above the surface 58 of the implant tray platform portion 41 higher than the anterior end 52 leaving the top wall 44 slanted upwardly away from the surface 58 from its anterior end 52 to its posterior end 50. The top wall 44 and the two opposite side walls 46 and 48 may be generally flat or can also be curved (not shown), and the two opposite side walls 46 and 48 may be either generally parallel to each other, or can slant inwardly toward each other from the posterior end to the anterior end of the keel (not shown.) The posterior end 50 thus defines a posterior keel opening 54 and the anterior end 52 thus defines an anterior keel opening 56 and a cavity or space S as well as a passageway is defined through the keel 42. When the implant tray platform with keel 40 is fitted to a bone implant site that has been prepared to snugly receive the keel 41, the close contact of the top wall 44 and side walls 46 and 48 with bone in the bone implant site will help to almost immediately secure the implant tray platform with keel 40 the bone implant site and prevent it from twisting or moving. Furthermore, over a relatively short period, bone will grow into a cavity inside of the keel 42 through the open ends 52 and 54 to thereby further lock the keel 42 in place at the implant site to better prevent movement of the implant tray platform with keel 40 at the bone implant site. The keel 62 and other portions of the implant tray platform with keel 40 that are to be locked into the bone preferably have surfaces that promote bone ingrowth and connection, such as by being surfaced to be rough. The keel 62 is preferably formed together (e.g. by casting, machining, etc.) as a single piece with the implant tray platform portion 41 and is made of strong material, including but not limited to titanium.

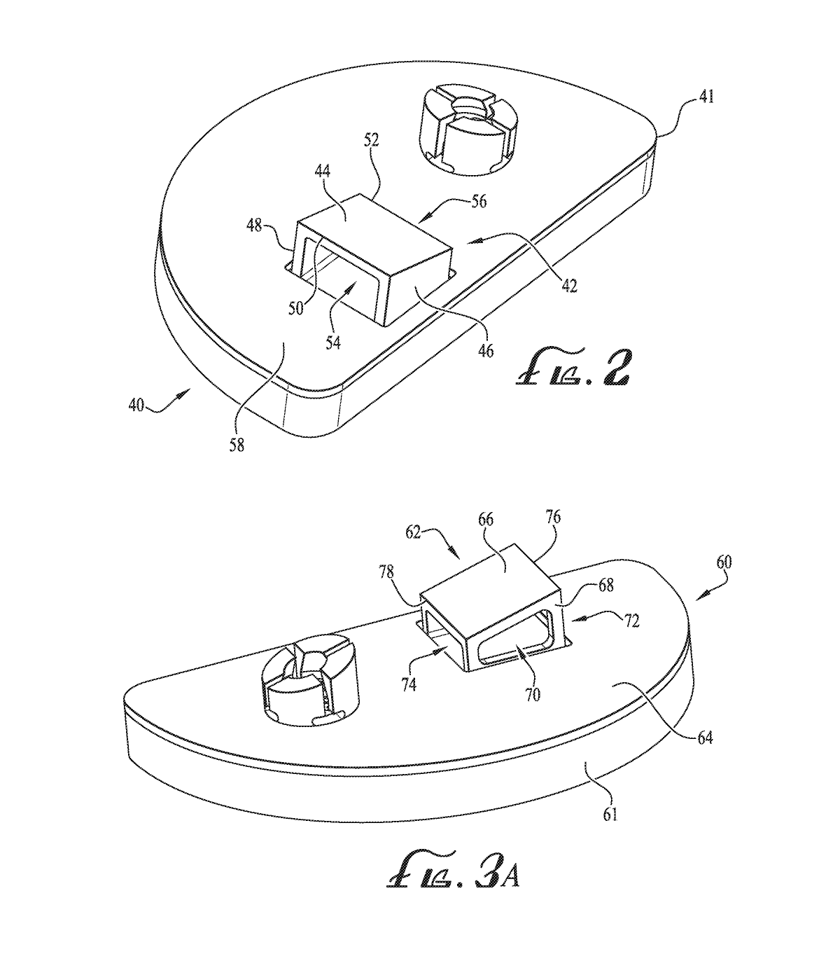

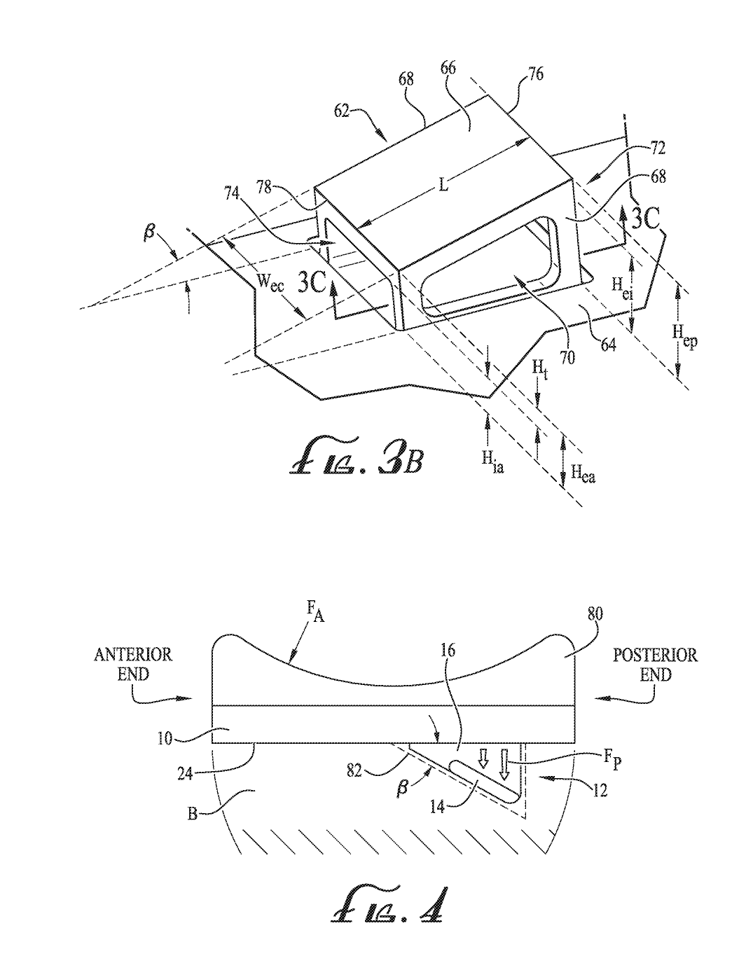

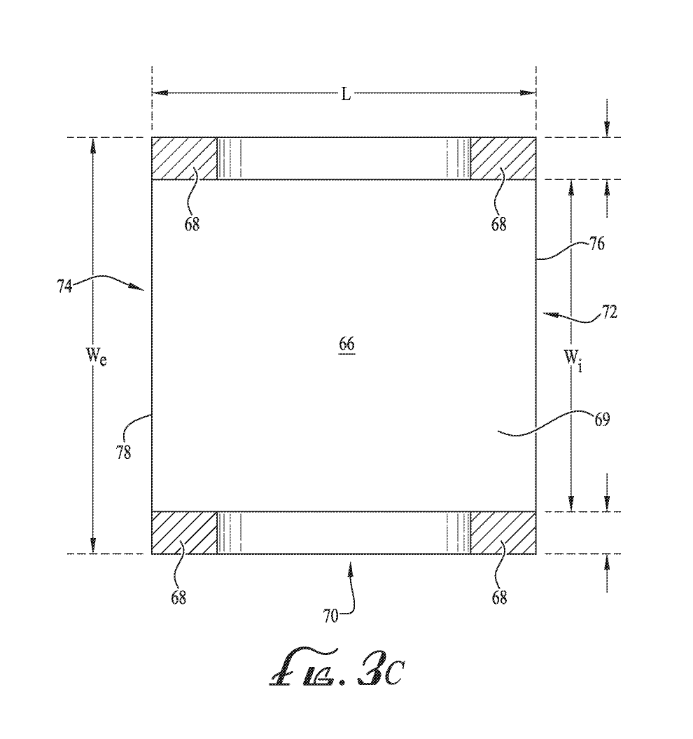

FIG. 3A is a perspective view of another exemplary implant tray platform with keel 60. FIG. 3B is a perspective detail view of keel 62. FIG. 3C is a cross-sectional view of the keel 62 through view lines 3C-3C of FIG. 3A. The keel 62 extends from a surface 64 of an implant tray portion 61 has a slanted top wall 66 one two opposed side walls 68, each with at least one opening 70 formed therethrough. The top wall 66 and the two opposite side walls 68 may be generally flat or can also be curved (not shown.) The two opposite side walls 68 may be either generally parallel to each other, or can slant inwardly toward each other from the posterior end to the anterior end of the keel (not shown.) The keel 62 has a higher posterior end 76 and a lower anterior end 78, and the posterior end 76 thus defines a posterior keel opening 72 and the anterior end 78 thus defines an anterior keel opening 74 and a cavity or space S as well as a passageway is defined through the keel 62. The slanted top wall 66 can be generally rectangular (but can have other shapes), and has a thickness Ht, a length L, an anterior external height Hₑₐ, an anterior internal height Hᵢₐ, a posterior external height Hₑₐ, a posterior internal height Hᵢₚ, an external width Wₑ and an internal width W. The slanted top wall 66 is preferably slanted up from anterior to posterior at angle 13 between about 10 to 20 degrees, and more preferably is slanted up by about 15 degrees. As shown, the openings 70 may be generally trapezoidal in shape, but can have other shapes, and there can be more than one window in the side walls 68. The keel 62 of FIGS. 3A, 3B, and 3C, by virtue of having openings 70 at its sides 68 as well as being open at its anterior end 74 and posterior end 72, provides additional advantages over the exemplary keel 42 of FIG. 2 because this keel design 62 will permit bone ingrowth through the opening 70 at the sides 68 in addition to at the open anterior end 74 and open posterior end 72, which bone ingrowth can eventually infill into a potential bone volume BV inside the keel 62 between the slanted top wall 66 and side walls 68, providing for tighter locking up of the keel 62 at the bone implant site. The top wall 66 is shown as being solid but can likewise have openings (not shown) formed therein if desired to permit bone ingrowth through the top wall 66 as well. By way of example, with a keel 62 having a top wall 66 thickness Hₜ of 0.0393″ (1.0 mm), a length L of 0.362″ (9.2 mm), an exterior width Wₑ of 0.394″ (10.0 mm), an interior width Wi of 0.315″ (8.0 mm), sidewalls 68 with a thickness of 0.0393″ (1.0 mm), distances Hₑₐ (0.109″ (2.8 mm)) and Hᵢₐ (0.071″ (1.8 mm)) from the outwardly facing surface and the inwardly facing surface, respectively, of the anterior end 78 of the top wall 66 to the surface 64 of the implant tray platform 61, distances Hₑₚ (0.203″ (5.1 mm)) and Hᵢₚ (0.161″ (4.1 mm)) from the inwardly facing surface and inwardly facing surface, respectively, of the posterior end 77 of the top wall 66 to the surface 64 of the implant tray platform 61, the cross-sectional surface area of the bottom of the top wall 66 would be about 0.1098 square inches (70.8 mm²), and the potential bone volume BV would be about 0.01268 cubic inches (207 mm³). Thus, when comparing both the cross-sectional areas and potential bone volumes of the prior art keel 12 of FIGS. 1A-C with the exemplary keel 62 of FIGS. 3A-C, the difference is quite significant. The prior art keel 12 has a cross-sectional area of about 0.0386 square inches (24.9 mm²) and potential bone volume of about 0.00372 cubic inches (60.9 mm³). On the other hand, the exemplary keel 62 of FIGS. 3A-C has a much larger cross-sectional area of about 0.1098 square inches (70.8 mm²) and potential bone volume BV of about 0.01268 cubic inches (207 mm³); this represents about a 185% increase in cross-sectional surface area and about a 240% increase in potential bone volume BV. The relevance of the cross-sectional surface area of the head/top wall of a keel and its potential bone volume BV is that increasing both will help prevent implant tray platforms from shifting as forces are exerted thereon, which is explained below with reference to FIGS. 4 and 5.

Turning to FIG. 4, there is shown a diagrammatic cutaway side view showing the prior art implant tray platform and keel 10 with a bearing portion 80 attached to a top of the implant tray platform portion 24, with the keel 12 extending into a space 82 formed in bone B at the implant site by having bone and other tissue removed to create a surface and space to receive the implant tray platform and keel 10. Over time, bone at the implant site will grow around the keel 12 of the implant tray platform and keel 10 and lock the keel 12 in place to help secure the implant tray platform and keel 10 at the implant site. When the bearing portion 80 is subjected to a force F_{A} that bears downwardly and forwardly on its anterior end, a counteracting force Fp will act on the keel 14 to hopefully prevent rocking and shifting of the implant tray platform and keel 10. It turns out that the amplitude of the counteracting force Fₚ will depend on several factors, including the cross-sectional surface area underside of the head 14 of the keel 12, and the potential bone volume BV captured between the head 14 of the keel and the surface 24 of the implant tray platform portion 24. With the arrow-shaped head 14 slanted at about 30 degrees, both the cross-sectional surface area underside of the head 14 and potential bone volume BV captured between the head 14 of the keel and the surface 24 of the implant tray platform portion 24 are relatively small, and the counteracting force Fₚ may not be able to counteract a strong force F_{A} that bears downwardly and forwardly on an anterior end of the implant. The average height of keel with an angled head is the sum of the height at the anterior end plus the height at the posterior end, divided by 2, and the potential bone volume BV will be this average keel height times the cross-sectional surface area of the head. Thus, in order to have a reasonably high average keel height, with a keel angled by 30 degrees, the posterior keel height must be relatively high since the anterior keel height will be relatively low. However, a high keel height may not be ideal for the following reason. In the process of creating surface(s) to support the implant, necrotic bone is removed to expose remaining healthy bone. Practitioners do their best to limit the amount of healthy bone removed since healthy bone supports the implant, and furthermore, implants wear out over time and must be replaced in revision surgeries. This requires that the old implant be removed, and that additional bone be removed at the implant site to provide a renewed surface for the replacement implant. It is desirable leave as much healthy and live bone during revision surgeries. In order to gain a reasonable high potential bone volume BV with the design of keel 12, the height of the keel 12 at its posterior end Hₑₚ needs to be relatively high to compensate for the relative low height of the keel 12 at its anterior end Hₑₐ. Thus, with the prior art implant tray platform and keel 10, the counteracting force Fₚ generated by its keel 12 might be lower than desired and the amount of bone that may need to be removed during revision surgeries may be on a higher side.

FIG. 5 is a diagrammatic cutaway side view showing the implant tray platform and keel 60 of FIG. 3A with a bearing portion 80 attached, with the keel 62 extending into a space 84 formed in bone B at the implant site by having bone and other tissue removed to create a surface and space to engage with the implant tray platform and keel 60. The space 84 is formed in the bone B by an angle Δ, which angle Δ is ideally be matched to the angle β of the top wall 66 relative to the surface 64 of the implant tray portion 61 so that there is a close fit of the of the space 84 to the top wall 66 of the keel. The space 84 in the bone B will preferably be likewise made to closely fit the side walls 68 of the keel 62. In practice, the space 84 in the bone B is created by use of a punch that pushes a portion of bone aside, and creates the angled glide path space 84. As can be seen, there will be an anterior portion 85 of the space 84 that is in front of the anterior end 78 of the keel 62 which remains unoccupied by the keel 62 when the implant tray platform and keel 60 are placed on the bone B. However, this anterior portion 85 is relatively small and bone will quickly grow into and fill this space, further preventing movement of the implant tray platform and keel 60 relatively to the bone B. When the bearing portion 80 is subjected to an anterior force F_{A}, the keel 62 can generate a resisting counteracting force Fp that help prevent rocking and shifting of implant tray platform and keel 60 relative to the bone B. As with the prior art implant, over time, bone grows and bone around the implant site will grow around the keel 62 of the implant tray platform and keel 60 and lock the keel 62 in place to help secure the implant tray platform and keel 60 at the implant site. When the bearing portion 80 is subjected to a force F_{A} that bears downwardly and forwardly on its anterior end, a counteracting force Fₚ will act on the keel 62 to help prevent rocking and shifting of the implant tray platform and keel 60. However, because of the significantly higher cross-sectional area and the significantly higher potential bone volume of the exemplary keel 62 of FIGS. 3A-C, the resisting counteracting force Fp is also higher. To repeat, the prior art keel 12 has a cross-sectional area of about 0.0386 square inches (24.9 mm²) and potential bone volume of about 0.00372 cubic inches (60.9 mm³). In comparison, the exemplary keel 62 of FIGS. 3A-C has a much larger cross-sectional area of about 0.1098 square inches (70.8 mm²) and potential bone volume BV of about 0.01268 cubic inches (207 mm³); this represents about a 185% increase in cross-sectional surface area and about a 240% increase in potential bone volume BV. The relevance of this higher cross-sectional surface area of the top wall of the keel and its potential bone volume BV is that the implant tray platforms will be more resistant to shifting as forces are exerted thereon. Another advantage of the less angled top wall 66 (angled between about 10 to 20 degrees) is that the average top wall height off the bone facing surface of the implant try can be made lower, which will help reduce the amount of bone which may need to be removed during revision surgery. Accordingly, the keel 62 of FIGS. 3A-C has advantages compared to prior art keels.

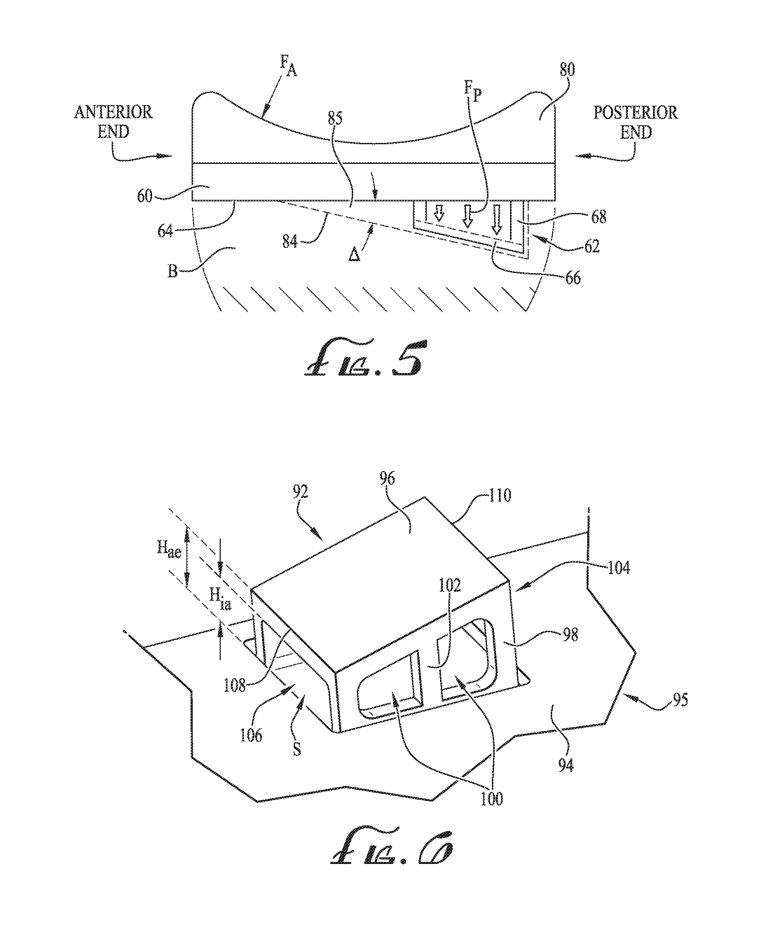

FIG. 6 is a detail perspective view showing another embodiment of a keel 92 that extends above a surface 94 of a tray portion 95. This keel 92 is very similar to the keel 62 of FIGS. 3A-3C, and includes a slanted top wall 96 that slats upwardly from an anterior edge 108 to a posterior edge. There are multiple side windows 100 in its side walls 98 that are divided by side bolsters 102, which can be included to further support the slanted top wall 96. The top wall 96 and the two opposite side walls 98 may be generally flat or can also be curved (not shown.) The two opposite side walls 98 may be either generally parallel to each other, or can slant inwardly toward each other from the posterior end to the anterior end of the keel (not shown.) The anterior end 108 of the keel has a posterior keel opening 106 and the posterior end 110 has a posterior keel opening 104. The keel 92 thus defines a cavity or space S as well as a passageway therethrough Like the keel 62 of FIGS. 3A-C, the top wall 96 is preferably slanted upwardly from anterior to posterior by between about 10 to 20 degrees, and more preferably by about 15 degrees and has similar advantages as discussed above.

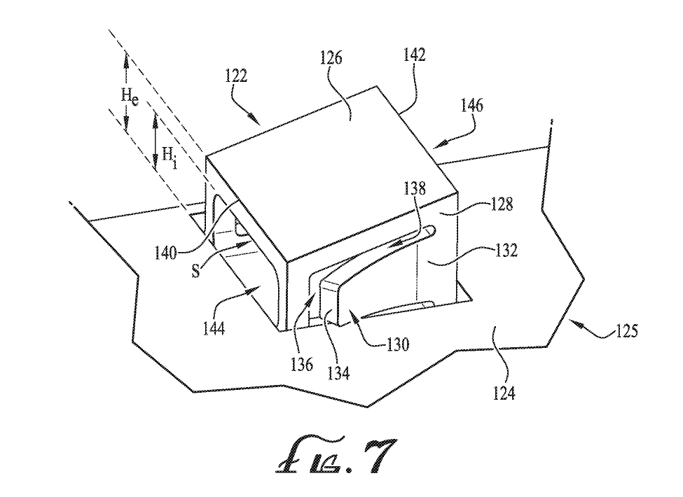

FIG. 7 is a detail perspective view showing yet another embodiment of a keel 122 with barbs 130 formed on its side walls 128. The barbs 130 are formed by reliefs 138 leaving a fixed end 132 and a free end 134 that extends away from the side walls 128, leaving a space 136. The keel 122 has a slanted top wall 126 that slants upwardly from its anterior edge 140 to its posterior edge 142 up from a surface 124 of a tray portion 125. The top wall 126 and the two opposite side walls 132 may be generally flat or can also be curved (not shown.) The two opposite side walls 128 may be either generally parallel to each other, or can slant inwardly toward each other from the posterior end to the anterior end of the keel (not shown.) The keel 122 preferably has an anterior keel openings 144 and a posterior keel opening 147, with the keel openings 144 and 146 available for bone ingrowth into the space S formed inside the keel 122. The barbs 130 have some flexibility so that when the keel is pushed into a space formed in the bone of an implant site (not shown), the barbs 130 will tend to flex out and make contact with the spaced formed in a prepared bone implant site and help retain the keel in place until bone can grow into and around the keel 122. The top wall 126 is preferably slanted upwardly from anterior to posterior by between about 10 to 20 degrees, and more preferably by about 15 degrees and this keel 122 has similar advantages as discussed above, with the additional feature of the barbs 130.

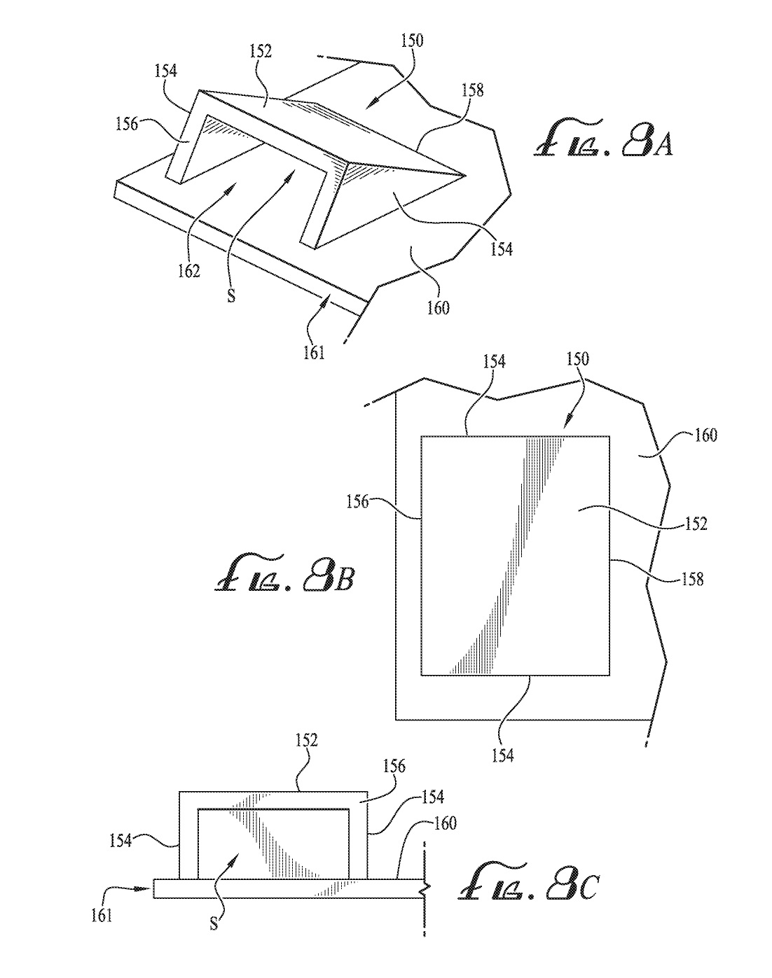

FIGS. 8A, 8B, and 8C are a posterior perspective view, a top view, and posterior end view, respectively, of another embodiment of an exemplary keel 150 which extend above a surface 160 of a tray portion 161. The keel 150 has a slanted top wall 152 joined to two opposing side walls 154 which slants down and intersect the surface 160 of tray portion 161 at an anterior end 158. The top wall 152 and the two opposite side walls 154 may be generally flat or can also be curved (not shown.) The two opposite side walls 154 may be either generally parallel to each other, or can slant inwardly toward each other from the posterior end to the anterior end of the keel (not shown.) At a posterior end 156 there is a posterior keel opening 162 that leads into a space S. This keel 150 will allow bone ingrowth through its posterior opening 162 and into the space S to help secure the keel 150 when implanted at a bone B implant site (not shown.) If desired, windows and/or barbs (not shown) can be formed in the side walls 154. This design is desirable when a particularly low profile keel is required with reasonably high bone volume and cross-sectional surface area to help retain the keel and associated tray portion 161.

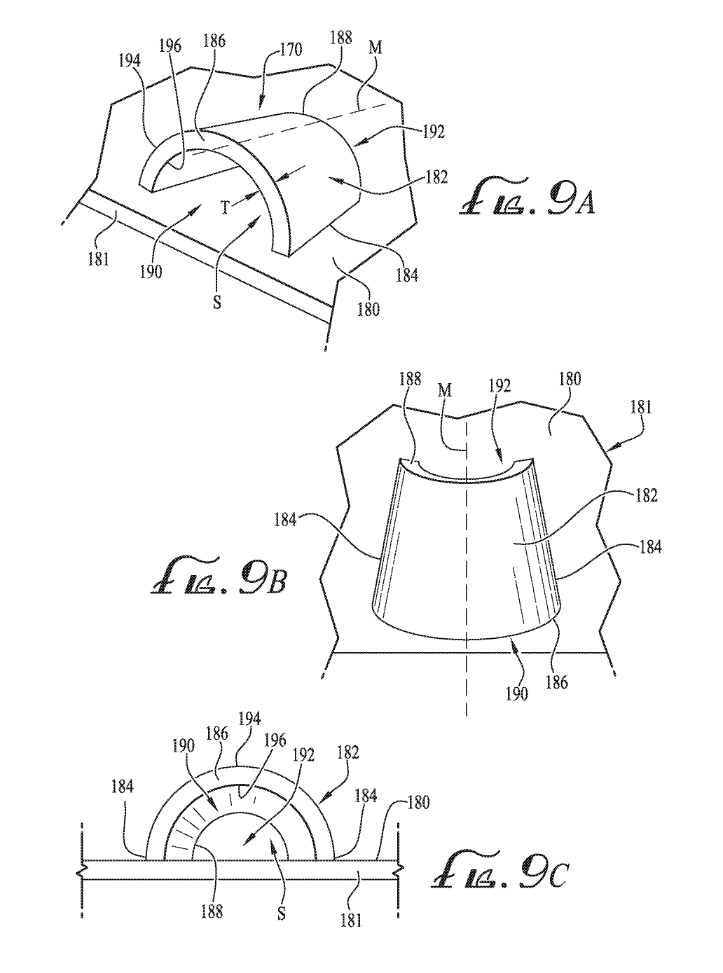

FIGS. 9A, 9B, and 9C are a posterior perspective view, a top view, and posterior end view, respectively, of still another embodiment of an exemplary keel 170 which extend above a surface 180 of a tray portion 181. The keel 170 has an arched wall 182 that arches up from two edges 184 that intersect the surface 180 of the tray portion 181. The arched wall 182 is in the form of a section of a truncated cone, and has a posterior end 186 and an anterior end 188, where the posterior end 186 is a larger arc section than the arc section of the anterior end 188. The arched wall 182 is preferably symmetrical along a midline M. There is a posterior keel opening 190 at the posterior end 186 and an anterior keel opening 192 at the anterior end 188. The arched wall 182 slopes downwardly at an angle of about 10 to 20 degrees from its posterior end 186 to its anterior end 188 towards the surface 190 of the tray portion 181 along the midline M and its two ends 184 angle inwardly. The arched wall 182 has an outer surface 194 and an inner surface 196 and the arched wall has a thickness “T”. There is a space “S” defined below the inner surface 196 of the arched wall 182 and the surface 180 of the tray portion 181 under the arched wall 182 which is available for bone ingrowth. Opening and/or barbs like shown in the embodiments of FIGS. 3A-C, and FIG. 7, respectively, can be formed on the arched wall 182 to aid in greater bone ingrowth into the keel 170 and enhanced retention of the keel 170 at an implant site.

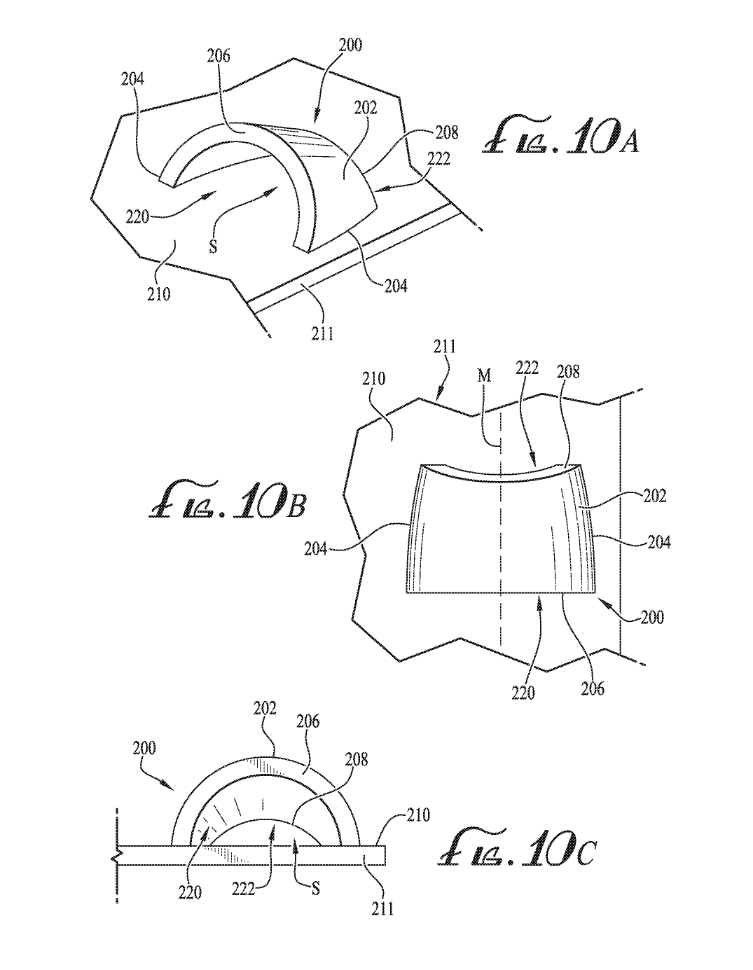

FIGS. 10A, 10B, and 10C are a posterior perspective view, a top view, and posterior end view, respectively, of yet another embodiment of an exemplary keel 200 which extend above a surface 210 of a tray portion 211. The keel 200 has an arched wall 202 that arches up from two edges 204 that intersect the surface 210 of the tray portion 211. The arched wall 202 is in the form of a section of a truncated portion of an ellipsoid, and has a posterior end 206 and an anterior end 208, where the posterior end 206 is a larger arc section than the arc section of the anterior end 208. The arched wall 202 is preferably symmetrical along a midline M and bows out and is convexly curved from the posterior end 206 to the anterior end 208. There is a posterior keel opening 220 at the posterior end 206 and an anterior keel opening 222 at the anterior end 208. The arched wall 202 generally slopes downwardly at an angle of about 10 to 20 degrees from its posterior end 206 to its anterior end 208 towards the surface 210 of the tray portion 211 along the midline M and its two edges 204 curve inwardly. The arched wall 202 has an outer surface 224 and an inner surface 226 and the arched wall has a thickness “T”. There is a space “S” defined below the inner surface 226 of the arched wall 202 and the surface 210 of the tray portion 211 under the arched wall 202 which is available for bone ingrowth. Opening and/or barbs like shown in the embodiments of FIGS. 3A-C, and FIG. 7, respectively, can be formed on the arched wall 202 to aid in greater bone ingrowth into the keel 200 and enhanced retention of the keel 200 at an implant site.

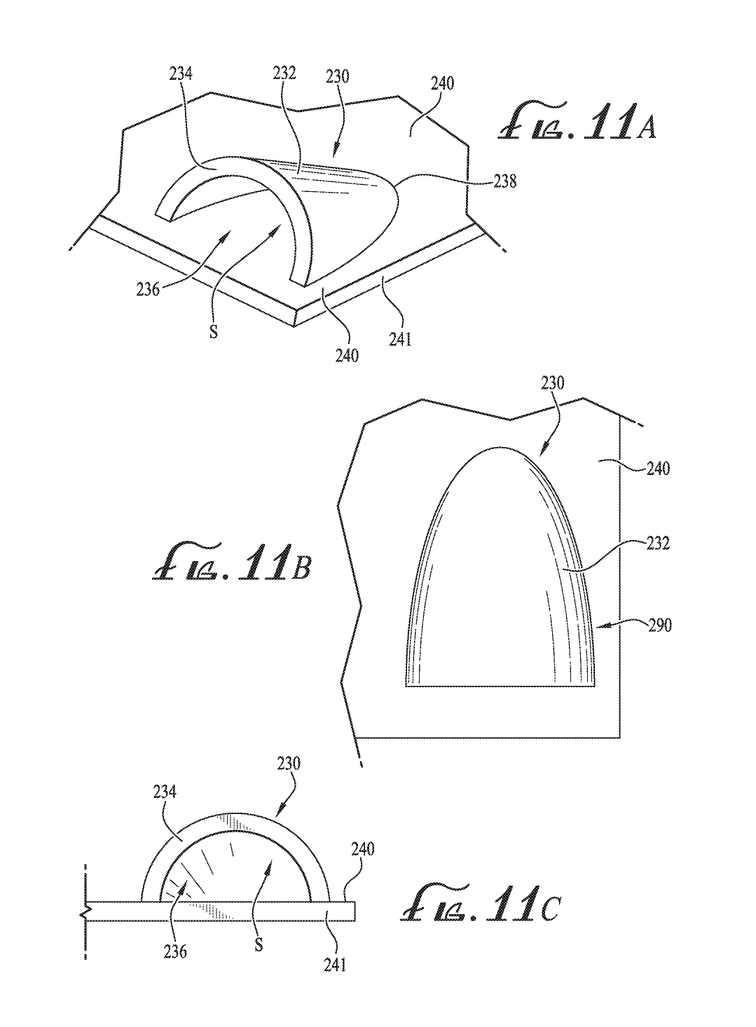

FIGS. 11A-C are a posterior perspective view, top view, and posterior end view, respectively, of another exemplary embodiment of a keel 230 and has an unraised arched wall 232 in the shape of a hollow truncated catenoid that extends above a surface 240 of a tray portion 241. It has an open posterior end 234 and has an open mouth 236. It drops down at its anterior end 238 to join the surface 240 of the tray portion 241. A space “S” is defined under keel wall 232 of the keel 230 which is available for bone ingrowth. Opening and/or barbs like shown in the embodiments of FIGS. 3A-C, and FIG. 7, respectively, can be formed on the arched wall 232 to aid in greater bone ingrowth into the keel 230 and enhanced retention of the keel 230 at an implant site.

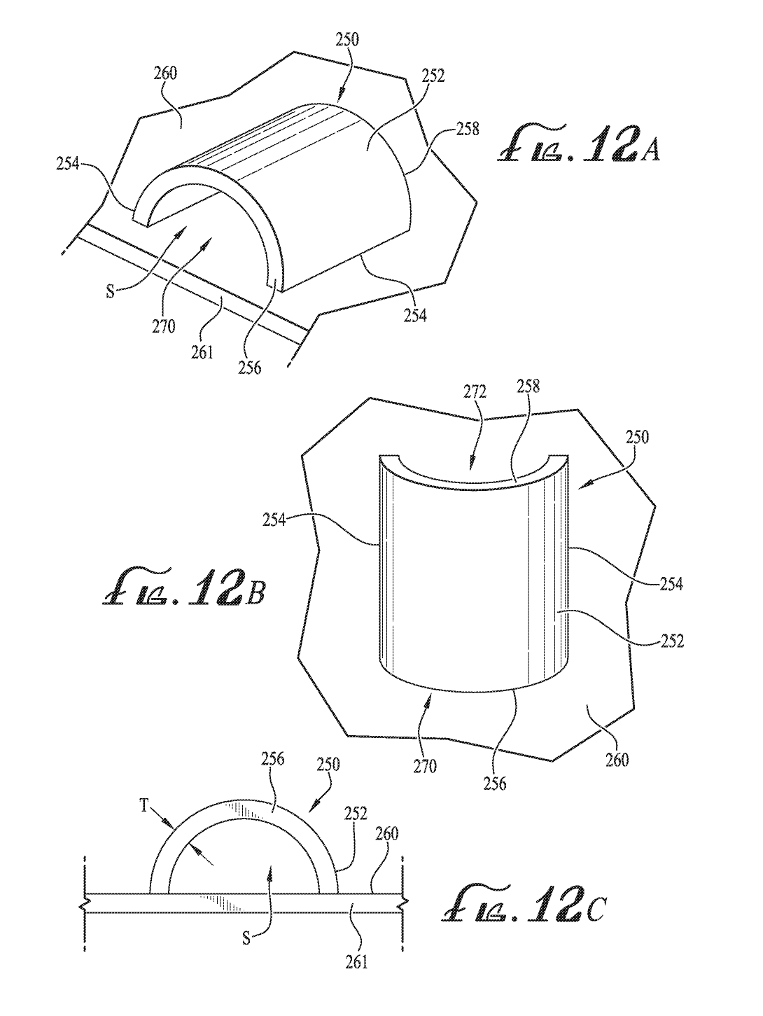

FIGS. 12A, 12B, and 12C are a posterior perspective view, a top view, and posterior end view of still another embodiment of an exemplary keel 250 which extend above a surface 260 of a tray portion 261. The keel 250 has an arched wall 252 that arches up from two ends 254 that join to the surface 260 of the tray portion 261. The arched wall 252 is in the form of a section of a hollow cylinder, and has a posterior end 256 and an anterior end 258, both of which are sized the same. There is an opening 270 at the posterior end 256 and an opening 272 at the anterior end 258. The arched wall 252 has an outer surface 274 and an inner surface 276 and the arched wall 252 has a thickness “T”. The thickness “T” can be, but need not be consistent throughout the arched wall 252. There is a space “S” defined below the inner surface 276 of the arched wall 252 and the surface 260 of the tray portion 261 which is available for bone ingrowth. Opening and/or barbs like shown in the embodiments of FIGS. 3A-C, and FIG. 7, respectively, can be formed on the arched wall 252 to aid in greater bone ingrowth into the keel 250 and enhanced retention of the keel 250 at an implant site. This keel 250 design, unlike the other design, is not sloped down from its posterior end 256 to its anterior end 258 but can be useful in applications where the keel 250 and tray portion 261 can be dropped straight down onto a bone implant site that has been prepared to receive the keel 250 and tray portion 261.

In embodiments, the keels can be formed together (e.g. by casting, machining, etc.) as a single piece with the implant tray platform portions and made of a strong material, such as titanium. However keels can be separate pieces that are permanently attached to the implant tray platform portions.

Any element in a claim that does not explicitly state “means” for performing a specified function or “step” for performing a specified function, should not be interpreted as a “means” or “step” clause as specified in 35 U.S.C. §112.

What is claimed is:

1. A keel for an orthopedic implant comprising:
a wall having a posterior end and an anterior end, the wall extending from a surface of a tray portion, the wall being at least partially upraised away from the surface of the tray portion at its posterior end defining a posterior keel opening, a bone ingrowth space being defined between the wall and the tray portion.

2. The keel of claim 1, wherein the anterior end of the wall is upraised from the surface of the tray portion and defines an anterior keel opening, and wherein a passageway is located in the keel from the posterior end to the anterior end.

3. The keel of claim 2, wherein the wall comprises two spaced apart side walls that extend from the surface of the tray portion and a top wall spanning a top of the two spaced apart side walls.

4. The keel of claim 3, wherein the top wall is slanted downwardly from its posterior end to its anterior end at an angle of about 10 to 20 degrees towards the surface of the tray portion.

5. The keel of claim 3, wherein the top wall is slanted downwardly from its posterior end to its anterior end at an angle of about 15 degrees towards the surface of the tray portion.

6. The keel of claim 3, wherein the two spaced apart side wall are generally flat and are generally parallel to each other, and wherein the top wall is generally flat.

7. The keel of claim 3, wherein there is at least one opening formed in each of the two side walls.

8. The keel of claim 3, wherein there is at least one barb extending outwardly from each of the two side walls.

9. The keel of claim 1, wherein the wall is an arched wall.

10. The keel of claim 1, wherein the wall is an arched wall generally in the shape of a section of a hollow truncated catenoid, and wherein the anterior end of the arched wall merges with the surface of the tray portion.

11. The keel of claim 2, wherein the wall is an arched wall, and wherein the posterior keel opening and the anterior keel opening are the same size.

12. The keel of claim 2, wherein the wall is an arched wall, and wherein the posterior keel opening is larger than the anterior keel opening.

13. The keel of claim 12, wherein the arched wall bows out and is convexly curved from its posterior end to its anterior end.

14. A keel for an orthopedic implant comprising:
a wall comprising two spaced apart side walls that extend from a surface of a tray portion and a top wall spanning a top of the two spaced apart side walls, the wall having a posterior end and an anterior end, the wall being upraised away from the surface of the tray portion at its posterior end to define a posterior keel opening and being upraised away from the surface of the tray portion at its anterior end to define an anterior keel opening, wherein the top wall is slanted downwardly from its posterior end to its anterior end at an angle of about 10 to 20 degrees towards the surface of the tray portion, and wherein a bone ingrowth space is defined between the wall and the tray portion.

15. The keel of claim 14, wherein the two spaced apart walls and the top wall are generally flat.

16. The keel of claim 14, wherein there is at least one opening or at least one barb formed in the side walls.

17. A keel for an orthopedic implant comprising:
an arched wall having a posterior end, an anterior end, which arched wall extends upwardly from a surface of a tray portion, the wall being at least partially upraised away from the surface of the tray portion at its posterior end to define a posterior keel opening, wherein a bone ingrowth space is defined between the wall and the tray portion.

18. The keel of claim 17, wherein the anterior end of the wall is upraised from the surface of the tray portion and defines an anterior keel opening, and wherein a passageway is located in the keel from the posterior end to the anterior end.

19. The keel of claim 17, wherein the wall is an arched wall generally in the shape of a section of a hollow truncated catenoid, and wherein the anterior end of the arched wall merges with the surface of the tray portion.

20. The keel of claim 17, wherein the wall is an arched wall, and wherein the posterior keel opening is larger than the anterior keel opening.

21. The keel of claim 11, wherein the arched wall bows out and is convexly curved from its posterior end to its anterior end.

22. The keel of claim 17, wherein the wall is an arched wall in the form of a section of a hollow cylinder, and wherein the posterior keel opening and the anterior keel opening are the same size.
